(19) United States
(12) Patent Application Publication
(10) Pub. No.: US 2015/0190517 A1
(43) Pub. Date: Jul. 9, 2015

(54) SYSTEMS AND METHODS FOR DELIVERING CROSS-LINKED HALYURONIC ACID INTO A PATIENT

(71) Applicants: Phi Nguyen, Houston, TX (US); Loc Phan, San Jose, CA (US); Bao Tran, Saratoga, CA (US); Thuan Nguyen, Houston, TX (US); Duy Bui, Ha Noi (VN)

(72) Inventors: Phi Nguyen, Houston, TX (US); Loc Phan, San Jose, CA (US); Bao Tran, Saratoga, CA (US); Thuan Nguyen, Houston, TX (US); Duy Bui, Ha Noi (VN)

(73) Assignee: MIBA MEDICAL INC., San Jose, CA (US)

(21) Appl. No.: 14/255,961

(22) PCT Filed: Apr. 12, 2012
(86) PCT No.: PCT/VN2013/000003

§ 371 Date: Mar. 24, 2015

(51) Int. Cl. A61K 47/36; A61K 31/436; A61K 31/573; A61K 31/337; A61F 2/12; A61K 31/167

(52) U.S. Cl. CPC A61K 47/36; A61F 2/12; A61K 31/167; A61K 31/573; A61K 31/337; A61K 31/436; A61F 2240/002; A61F 2250/0003; A61F 2250/0067; A61B 2019/507

(57) ABSTRACT

Systems and methods are disclosed for cosmetic augmentation by forming a biocompatible cross-linked polymer having a multi-phase mixture with a predetermined controlled release of selected pharmaceutical substance to modulate soft tissue response to the polymer; injecting the mixture into a patient and during or after injection, cross-linking the polymer in the patient; and augmenting soft tissue with the biocompatible cross-linked polymer.

This application claims priority to Provisional Application Ser. 61/722,221 filed Nov. 4, 2012 and a national conversion of PCT Application Serial PCT/VN2013/000001 filed Apr. 12 2013, PCT/VN2013/000002 filed Apr. 12 2013, PCT/VN2013/000003 filed on Apr. 12 2013, PCT/VN2013/000004 filed Apr. 12 2013, and PCT/VN2012/000008 filed Dec. 17 2012, the contents of which are incorporated by reference.

### BACKGROUND

The present invention relates to biodegradable hyaluronic acid filler compositions for soft tissue implants such as dermal fillers or breast, butt, or body implants.

Everyone wants healthy, younger-looking skin. Having a smoother, youthful appearance can make a person look years younger and feel beautiful and self-assured. Injectable dermal implants are a popular solution to a wide variety of facial contour defects from lip augmentation to plumping up depressed scars. Often used as tissue replacement for victims of serious accidents, injectable dermal implants are very effective in cosmetic surgery procedures such as lip augmentation and scar removal.

In a parallel trend, millions of women have undergone breast, butt, or body augmentation and reconstruction in the past few decades. Most women choose augmentation to enhance the size and shape of one or both breast, butt, or body parts for personal or aesthetic reasons. In contrast, women who undergo a reconstruction procedure want to reconstruct a breast, butt, or bodypart that has been removed, typically for health reasons, such as tumor removal. The reconstruction procedure may vary from a modified radical mastectomy (removal of the underlying muscle as well as the breast, butt, or body part), to a simple mastectomy (removal of one breast, butt, or body part), to a bilateral mastectomy (removal of both breast, butt, or body parts) or to a lumpectomy (removal of a portion of the breast, butt, or body part). In either augmentation or reconstruction, the modality intimates the surgical implantation of a breast, butt, or body prosthesis (implant).

Conventional implants for treating breast, butt, or body augmentation or reconstruction include a shell or envelope that is filled with a filler composition, for example, silicone gel, saline solution, or other suitable filler. It is desirable that the filler have lubricating properties to prevent shell abrasion, remain stable over long periods of time, be non-carcinogenic and non-toxic, and have physical properties to prevent skin wrinkling, capsular contracture formation, and implant palpability.

While breast, butt, or body implants containing silicone-gel as filler materials are widely used for breast, butt, or body augmentation or reconstruction, a variety of potential disadvantages have been recognized with respect to the stabilization of the implants and the immune system. First, the silicone gel-filled implants have a tendency to leak. In 1992, the FDA issued a voluntary moratorium on silicone gel-filled implants due to public health concerns regarding the potential link between leaking silicone gel-filled implants and autoimmune diseases. To date, the long term effect of silicone-gel on the immune system is still unknown. Second, the leaking of the implants necessitates the need for additional surgeries for removal or repair of the implants. Third, the silicone-gel as a filling material has a greater density than saline or natural tissues which may cause recipients back pain. Fourth, silicone is a permanent filler composition and when it leaks, it can travel though out the body and can cause unwanted hard nodular formations if left untreated. Lastly, the silicone-gel implant does not mimic the touch and feel of a real breast, butt, or body even though it offers a more realistic feel than saline as a filler material.

Many plastic surgeons turned to saline as an answer to silicone-gel problems. Several implants which use saline are known and were found to be advantageous over silicone-gel for several reasons. Saline has a lower density than silicone-gel causing less strain on recipients' backs. In addition, if the implant leaks, the saline solution is non-toxic providing a more tolerated and safer implant than those containing silicone-gel.

However, while the saline implant offer significant advantages over the silicone-gel implant, various problems have been encountered. Implants using saline are disadvantageous in that they frequently result in capsular contraction, a phenomenon where the body forms a lining of fibrous tissue encapsulating the breast, butt, or body implant and the resulting capsule tightens and squeezes the implant. Symptoms range from mild firmness and mild discomfort to severe pain, distorted shape, palpability of the implant, and/or movement of the implant. Additional surgery may be needed in cases where pain and/or firmness are severe. This surgery ranges from removal of the implant capsule tissue to removal and possibly replacement of the implant itself. There is no guarantee that capsular contracture will not occur after these additional surgeries.

Saline implants may have to be removed and replaced periodically for other reasons—they fracture or they deflate. Saline, because it is less viscous than silicone-gel, settles in the bottom portion of the implant when the recipient is upright. This leaves the upper portion of the implant prone to excessive folding or wrinkling, causing stress fracturing of the shell at the fold points. Furthermore, the saline-filled implants have a tendency to drain gradually in about ten years. Barring any deflation or rupture complications, saline as a filler for breast, butt, or body implants produces an unnatural feel and look to the implant.

U.S. Pat. No. 6,881,226 discloses a breast, butt, or body implant having at least an outer shell which is composed of a resorbable material. The implant, which can be formed entirely of bioresorbable material such as collagen foam, is sized and shaped to replace excised tissue. The implant supports surrounding tissue upon implantation, while allowing for in-growth of fibrous tissue to replace the implant. According to various alternative embodiments, the implant is elastically compressible, or can be formed from self-expanding foam or sponges, and can be implanted through a cannula or by injection, as well as by open procedures. The implant can carry therapeutic and diagnostic substances.

In response to the failures of saline and silicone-gel implants, there have been a number of attempts to make a prosthesis filled with a non-toxic filler that that mimics the shape and feel of a natural breast, butt, or body provided by silicone-gel yet is safe to the immune system like saline. Other attempts to provide a safe filler material include polyethylene glycol. However, the triglyceride oil or honey fails to provide an implant that is aesthetically pleasing and also duplicates the touch and feel of a natural breast, butt, or body due to the low viscosity of the fillers. Due to the limited options and the inadequacy of current fillers to achieve the desired results, there is a need for safe and efficacious fillers.

### SUMMARY

In one aspect, systems and methods are disclosed for cosmetic augmentation by forming a biocompatible cross-linked polymer having a multi-phase mixture with a predetermined controlled release of selected pharmaceutical substance to modulate soft tissue response to the polymer; injecting the mixture into a patient and during or after injection, cross-linking the polymer in the patient; and augmenting soft tissue with the biocompatible cross-linked polymer.

In another aspect, systems and methods are disclosed for breast, butt, or body implants by forming a biocompatible cross-linked polymer having a multi-phase mixture with a predetermined controlled release of selected pharmaceutical substance to modulate soft tissue response to the polymer; injecting the mixture into a patient and during or after injection, cross-linking the polymer in the patient; filling a semi-permeable shell with the pharmaceutical substance; and augmenting soft tissue with the biocompatible cross-linked polymer.

### BRIEF DESCRIPTION OF THE DRAWINGS

FIG. 1 shows an exemplary block diagram of a computer controlled hyaluronic acid (HA) injector system that cross-links the HA while the drug is injected into the body.

FIGS. 2-3 shows an exemplary manual hyaluronic acid (HA) injector system that cross-links the HA while the drug is injected into the body.

FIG. 4 shows an exemplary breast, butt, or body implant delivery system.

FIG. 5 shows an exemplary process to inject and cross-link materials at the same time.

FIG. 6 shows another exemplary process to inject and cross-link materials at the same time.

### DESCRIPTION

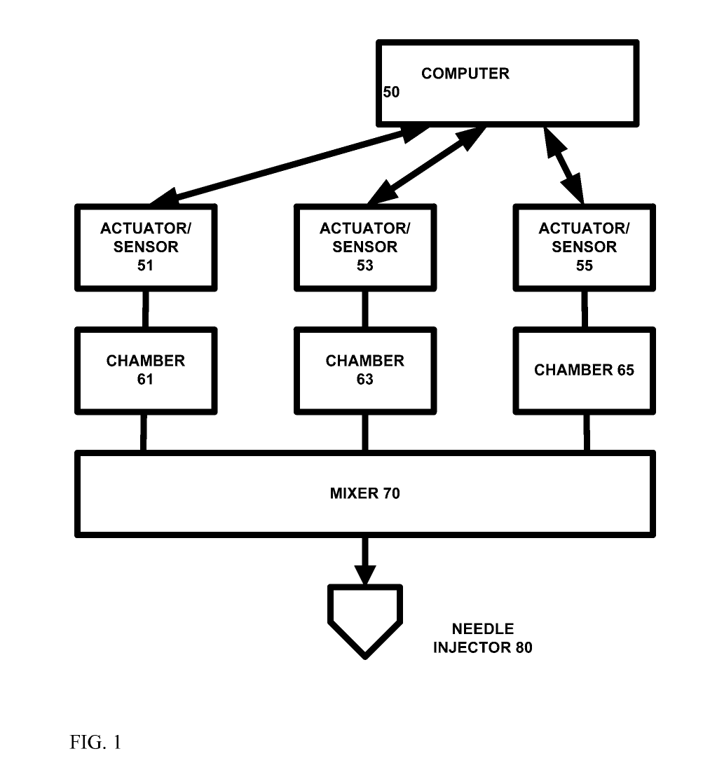

FIG. 1 shows an exemplary block diagram of a hyaluronic acid (HA) injector system that cross-links the HA while the drug is injected into the body. As shown therein, a triple cartridge with parallel containers each housing one of three flowable components to be mixed when desired in a static mixer and which terminates in an outlet tip from which the components mixed by the static mixer are expelled. The static mixer may be separable from and attached to the containers or chambers in a manner known per se. The containers, usually made of a plastics material, are joined by a bridge defining an outlet in which the two components are separated by an internal dividing wall to maintain the components separate and unmixed until they reach inlet of the static mixer for mixing therein. In a conventional manner, the static mixer, again usually of a plastics material, comprises a static mixer element housed in an elongate member extending from attachment to the outlet to outlet tip. Also the static mixer element comprises an axially extending serial plurality of alternating oppositely oriented helically twisted mixer blades which act in concert to efficiently and thoroughly mix the separate components as they flow through the static mixer 6 from the outlet to the outlet tip. Pistons or motorized actuators are operated simultaneously by a suitable mechanism (not shown) with the cartridge being retained by the back plate, to dispense the components simultaneously from the containers through the outlet and static mixer to the outlet tip. In this embodiment, the actuators are controlled by a computer for precise mixing and delivery as desired. Moreover, a plurality of outlets can be provided so that a plurality of patient areas can be injected in parallel.

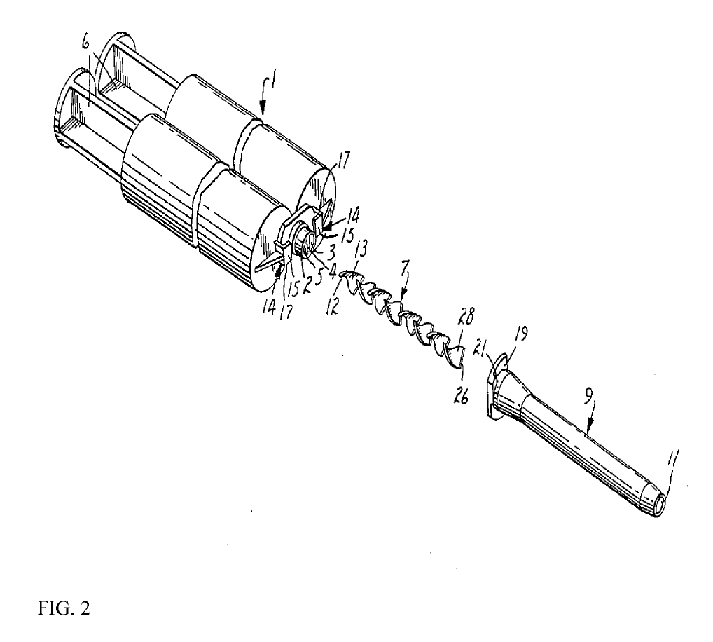

Referring now to FIG. 2, there is shown an exploded view in perspective of a static mixing device for forming cross-linked HA as it is injected into the patient. Although illustrated as a dual chamber device, syringe 1 has two or three parallel internal chambers, each of which is intended to be filled with a cross-linked material such as DVS, a filler material such as hyaluronic acid, and a catalyst such as sodium bicarbonate solute. The chambers in syringe 1 are separated by barrier 4. When a pair of plungers 6 are forced into the chambers in syringe 1, the contents of the syringe exit via outlet 2 through outlet passages 3 and 5, flow through static mixing element 7 and exit conduit 9, and are intimately mixed to form a homogeneous mass which will rapidly polymerize following expulsion from outlet 11 of exit conduit 9. Static mixing element 7 is prevented from being expelled during use from the outlet end of exit conduit 9 by a suitable constriction in the inside diameter of exit conduit 9 proximate its outlet end.

In one embodiment, maximum efficiency of mixing is obtained by insuring that the inlet end 12 of the first mixing blade 13 of static mixing element 7 is generally perpendicular to the plain of contiguity between the two resin streams exiting syringe 1 through exit passages 3 and 5. Such perpendicular orientation is obtained using a locating tang in exit conduit 9, which locating tang serves to orient static mixing element 7 with respect to syringe 1.

Rotational alignment of exit conduit 9 with respect to syringe 1 is obtained using a suitable mounting means (e.g., a bayonet mount). Bayonet locking tabs 14 have locking prongs 15 and stop surfaces 17. Exit conduit 9 has locking ramps 19 and stop surfaces 21. Exit conduit 9 is mounted on syringe 1 by centering the inlet of exit conduit 9 over outlet 2 of syringe 1, while aligning exit conduit 9 so that it can be pushed between bayonet locking tabs 14. Exit conduit 9 is then inserted firmly over outlet 2, and rotated approximately 90° clockwise (as viewed from the exit end of the conduit) so that locking ramps 19 are wedged between locking prongs 15 and the main body of syringe 1, and stop surfaces 17 engage stop surfaces 21.

When so mounted, exit conduit 9 is fixably rotationally aligned with respect to syringe 1. In addition, through locating means described in more detail below, static mixing element 7 is fixably rotationally aligned with respect to exit conduit 7 and syringe 1. Static mixing element 7 and exit conduit 9 are firmly attached to syringe 1, but can be readily removed and discarded after use by rotating exit conduit 9 approximately 90° counterclockwise (as viewed from the exit end of the conduit) and pulling exit conduit 9 away from syringe 1.

Syringe 1, exit nozzle 2, exit passages 3 and 5, barrier 4, plungers 6, static mixing element 7, exit conduit 9, inlet edge 12, first mixing blade 13, bayonet locking tabs 14, and locking prongs 15 are as in FIG. 1. Static mixing element 7 is rotationally aligned within exit conduit 9 by one or more guides proximate the outlet end of exit conduit 9. Guides 24 and 25 are small inward projections in the bore of exit conduit 9, and have a “fish mouth” appearance when viewed in perspective. When viewed in isolation, locking guides 24 and 25 each resemble the nib of a fountain pen.

When static mixing element 7 is inserted into the inlet end of exit conduit 9, and pushed toward the outlet end of exit conduit 9, guides 24 and 25 serve to rotationally align static mixing element 7 within exit conduit 9. When leading edge 26 of the final mixing blade 28 of static mixing element 7 approaches the outlet end of exit conduit 9, guides 24 and 25 cause static mixing element 7 to rotate about its long axis until leading edge 26 abuts edge surface 24a of guide 24 or edge surface 25a of guide 25.

When a static mixing element is inserted sufficiently far into exit conduit 9 to strike cusp 33, the leading edge of the static mixing element is deflected by cusp 33 toward edge surface 24a or toward edge surface 24b, thereby providing the desired rotational alignment. Depending upon whether the static mixing element abuts against edge surface 24a or 24b of guide 24 (and against corresponding edge surface 25b or 25a of guide 25), the final orientation of the static mixing element will be in one of two positions, each of those positions being 180° of rotation apart from the other. Each position is equally acceptable as a means for optimizing the efficiency of the first blade of the static mixing element, since in either position the first mixing element will intersect the incoming streams of resin at an approximate right angle to the plane of contiguity between the incoming streams and subdivide the incoming streams equally.

Although FIG. 2 shows two chambers 4, one embodiment provides three chambers 4: a first chamber containing a cross-linking material such as DVS, a second chamber containing hyaluronic acid (HA), and a third chamber containing a catalyst such as sodium bicarbonate solution.

The inner content 6 of the implant is a composition that is composed mainly of hyaluronic acid. The term “hyaluronic acid” is used in literature to mean acidic polysaccharides with different molecular weights constituted by residues of D-glucuronic and N-acetyl-D-glucosamine acids, which occur naturally in cell surfaces, in the basic extracellular substances of the connective tissue of vertebrates, in the synovial fluid of the joints, in the endobulbar fluid of the eye, in human umbilical cord tissue and in cocks' combs. The term “hyaluronic acid” is in fact usually used as meaning a whole series of polysaccharides with alternating residues of D-glucuronic and N-acetyl-D-glucosamine acids with varying molecular weights or even the degraded fractions of the same, and it would therefore seem more correct to use the plural term of “hyaluronic acids”. The singular term will, however, be used all the same in this description; in addition, the abbreviation “HA” will frequently be used in place of this collective term. HA can also be defined as an unsulphated glycosaminoglycan composed of repeating disaccharide units of N-acetylglucosamine (GIcNAc) and glucuronic acid (GlcUA) linked together by alternating beta-1,4 and beta-1,3 glycosidic bonds. Hyaluronic acid is also known as hyaluronan, hyaluronate, or HA. The terms hyaluronan and hyaluronic acid are used interchangeably herein. More details on how to make the HA are discussed in commonly owned, co-pending application Ser. No. 13/353,316, filed Jan. 18, 2012, and entitled “INJECTABLE FILLER,” the content of which is incorporated by reference.

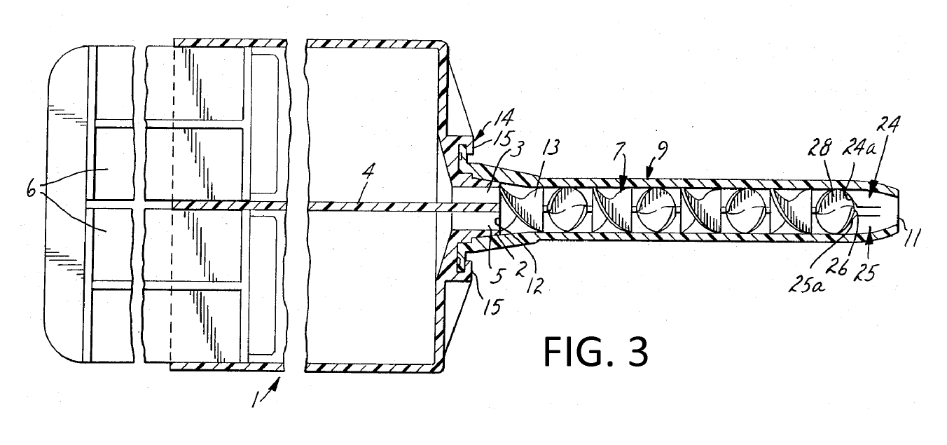

The injectors of FIG. 1 or FIGS. 2-3 can be used to inject HA for dermal filling or for filling a breast, butt, or body implant, as shown in FIG. 4.

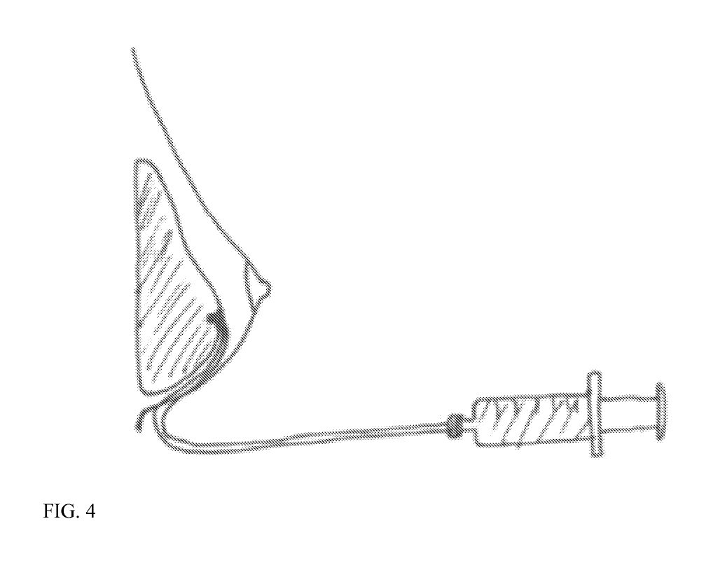

FIG. 5 shows an exemplary process to inject and cross-link materials at the same time. The process includes forming a biocompatible cross-linked polymer having a multi-phase mixture each in a separate container with a predetermined controlled release of selected pharmaceutical substance to modulate soft tissue response to the polymer (702). Next, the process includes injecting the mixture as separate phases into the body and mixing the mixture during the injection process to cause cross-linking of the multiphase mixture (703). The process then fills a semi-permeable shell with the pharmaceutical substance (704). The process then augments soft tissue with the biocompatible cross-linked polymer (706)

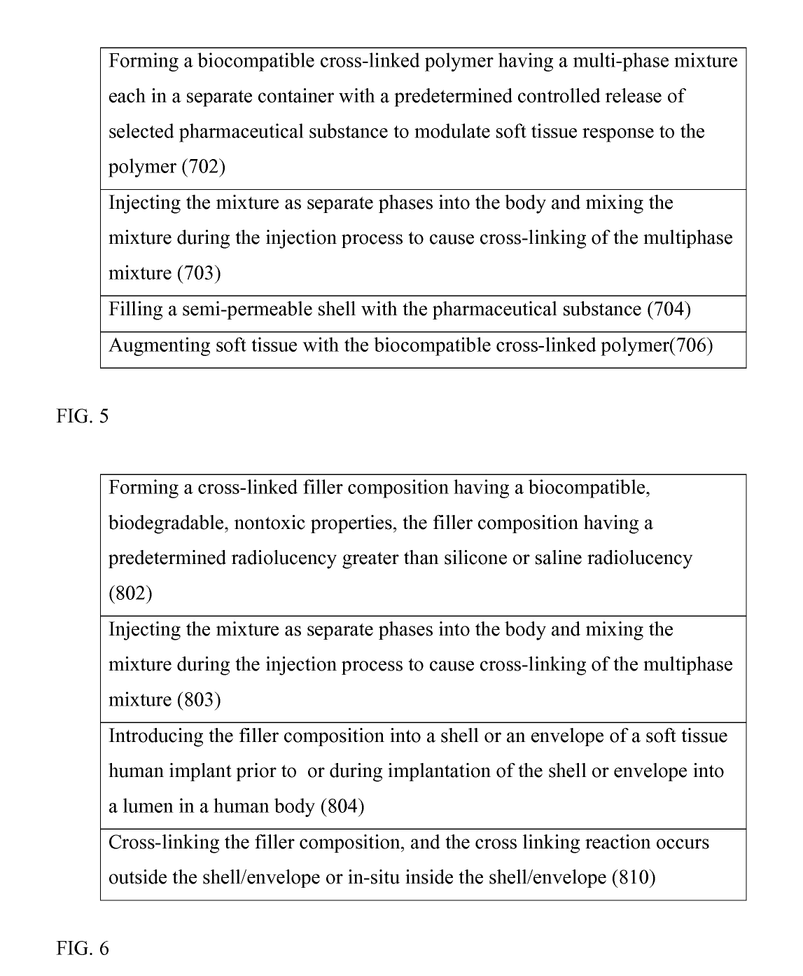

FIG. 6 shows another exemplary process to inject and cross-link materials at the same time. The process includes forming a cross-linked filler composition having a biocompatible, biodegradable, nontoxic properties, the filler composition having a predetermined radiolucency greater than silicone or saline radiolucency (802). The process also injects the mixture as separate phases into the body and mixing the mixture during the injection process to cause cross-linking of the multiphase mixture (803). The filler composition with HA and cross linking materials is introduced into a shell or an envelope of a soft tissue human implant prior to or during implantation of the shell or envelope into a lumen in a human body (804). The cross-linking the filler composition occurs, and the cross linking reaction occurs outside the shell/envelope or in-situ inside the shell/envelope (810)

With certain HAs, the cross linking of the HA external to the shell can cause the cross-linked gel to become hardened and thus the HA may not be inserted into the shell easily with desired properties. A reversible cross-linking system can be used in one embodiment, where the cross links will be labile at extreme pH values, and at physiological pH, the cross-links become fixed. Two product streams can enter the shell, one is the product at an altered pH state and the other is the PBS, the neutralizer.

Gelling by either bioresponsive self-assembly or mixing of binary crosslinking systems, these technologies are useful in minimally invasive applications as well as drug delivery systems in which the sol-to-gel transition aids the formulation's performance. Moreover, not only does the chemical nature of the crosslinking moieties allow these systems to perform in situ, but they contribute dramatically to the mechanical properties of the hydrogel networks. For example, reversible crosslinks with finite lifetimes generate dynamic viscoelastic gels with time-dependent properties, whereas irreversible crosslinks form highly elastic networks.

The intrinsic properties of in situ forming gels add a new dimension of flexibility to large space augmentation such as that of the breast, body or the buttock. While the silicone filled shell gives the feel and touch of native tissue, the long term health and legal complications associated of foreign body reaction and biocompatibility cannot be avoided. The over the lifetime of the implant, the fact that silicone fluid finding its way to the tissue on the outside of the shell is a kinetic eventuality. Saline filled shell has been a reluctant alternative because its feel and aesthetic affect are far from natural. The best of both worlds alternative might be found in a native material such as hyaluronic acids. The required properties might be best satisfied in an in situ crosslinked hyaluronic acid, or ex situ crosslinked hyaluronic acid or super high molecular weight linear (uncrosslinked) hyaluronic acids.

The following are examples of in situ crosslinking method for hyaluronic acids:

1. Hyaluronic acids, hydrazide and aldehyde:

Doubly crosslinked networks composed of HA microgels and crosslinked hydrogels with tunable is coelasticity in the relevant frequency range have been proposed for vocal fold healing. These partially monolithic and partially living materials feature divinylsulfone-crosslinked HA particles that have been oxidized with periodate to produce surface aldehyde functionalities.

A derivative of hyaluronic acid (HA), comprising the steps of:

1.1. forming an activated ester at a carboxylate of a glucuronic acid moiety of hyaluronic acid;

1.2. substituting at the carbonyl carbon of the activated ester formed in step 1.1

1.3. a side chain comprising a nucleophilic portion and a functional group portion; and

1.4. forming a cross-linked hydrogel from the functional group portion of the hyaluronic acid derivative in solution under physiological conditions wherein the forming of a cross-linked hydrogel is not by photo-cross-linking.

2. Hyaluronic acid, dextran by forming a hydrazine

3. Functionalization of hyaluronic acid (HA) with chemoselective groups enables in situ formation of HA-based materials in minimally invasive injectable manner. One embodiment of HA modification with such groups primarily rely on the use of a large excess of a reagent to introduce a unique reactive handle into HA and, therefore, are difficult to control. FIG. 9 shows another embodiment with a protective group strategy based on initial mild cleavage of a disulfide bond followed by elimination of the generated 2-thioethoxycarbonyl moiety ultimately affording free amine-type functionality, such as hydrazide, aminooxy, and carbazate. Specifically, new modifying homobifunctional reagents may be synthesized that contain a new divalent disulfide-based protecting group. Amidation of HA with these reagents gives rise to either one-end coupling product or to intra/intermolecular cross-linking of the HA chains. However, after subsequent treatment of the amidation reaction mixture with dithiothreitol (DTT), these cross-linkages are cleaved, ultimately exposing free amine-type groups. The same methodology was applied to graft serine residues to the HA backbone, which were subsequently oxidized into aldehyde groups. The strategy therefore encompasses a new approach for mild and highly controlled functionalization of HA with both nucleophilic and electrophilic chemoselective functionalities with the emphasis for the subsequent conjugation and in situ cross-linking. A series of new hydrogel materials were prepared by mixing the new HA-aldehyde derivative with different HA-nucleophile counterparts. Rheological properties of the formed hydrogels were determined and related to the structural characteristics of the gel networks. Human dermal fibroblasts remained viable while cultured with the hydrogels for 3 days, with no sign of cytotoxicity, suggesting that the gels described in this study are candidates for use as growth factors delivery vehicles for tissue engineering applications.

4. The gelation is attributed to the Schiff base reaction between amino and aldehyde groups of polysaccharide derivatives. In the current work, N-succinyl-chitosan (S-CS) and aldehyde hyaluronic acid (A-HA) were synthesized for preparation of the composite hydrogels.

5. FIG. 10 shows injectable hyaluronic acid (HA) hydrogels cross-linked via disulfide bond are synthesized using a thiol-disulfide exchange reaction. The production of small-molecule reaction product, pyridine-2-thione, allows the hydrogel formation process to be monitored quantitatively in real-time by UV spectroscopy. Rheological tests show that the hydrogels formed within minutes at 37° C. Mechanical properties and equilibrium swelling degree of the hydrogels can be controlled by varying the ratio of HA pyridyl disulfide and macro-cross-linker PEG-dithiol. Degradation of the hydrogels was achieved both enzymatically and chemically by disulfide reduction with distinctly different kinetics and profiles. In the presence of hyaluronidase, hydrogel mass loss over time was linear and the degradation was faster at higher enzyme concentrations, suggesting surface-limited degradation.

Other Examples include:

A. To produce a crosslinked hyaluronic acid filler composition by in-situ cross linking Using DivinylSulfone to fill a 200 mL silicone shell:

| | | | |
|---|---|---|---|
| 1. | Hyaluronic Acid (2M Dalton) | l.5 | g |
| 2. | NaOH (0.2N) | 50 | mL |
| 3. | Combine the two and mix until completely dissolved | | |
| 4. | Inject this hyaluronic acid/NaOH mixture into | 35 | μL |
| | the silicone shell | | |
| 5. | Divinylsulfone | 150 | μL |
| 6. | PBS (phosphate buffered saline) | 150 | mL |
| 7. | Thoroughly mix the PBS and DVS | | |
| 8. | Inject the PBS/DVS into the shell | | |
| 9. | Mix vigarously together for homogeneous | | |
| | crosslinking reaction | | |
| 10. | Neutralize using an appropriate amount of acid | | |
| | such as hydrochloric acid with the pH be monitored. | | |
| 10. | Use appropriately | | |

B. To produce a crosslinked hyaluronic acid filler composition by in-situ cross linking Using 1,4-butane dioldiglycidyl ether (BDDE) to fill a 200 mL silicone shell:

| | | | |
|---|---|---|---|
| 1. | Hyaluronic Acid (2M Dalton) | 2.0 | g |
| 2. | NaOH (0.2N) | 50 | mL |
| 3. | Combine the two and mix until completely dissolved | | |
| 4. | Inject this hyaluronic acid/NaOH mixture into the | 40 | μL |
| | silicone shell | | |
| 5. | 1,4-butane dioldiglycidyl ether | 150 | μL |
| 6. | PBS (phosphate buffered saline) | 150 | mL |
| 7. | Thoroughly mix the PBS and BDDE | | |
| 8. | Inject the PBS/BDDE into the shell | | |
| 9. | Mix vigarously together for homogeneous crosslinking | | |
| | reaction | | |
| 10. | Neutralize using an appropriate amount of acid such as | | |
| | hydrochloric acid with the pH be monitored. | | |
| 11. | Use appropriately | | |

The viscosity of these polymers could be controlled by using its pH properties. The low viscosity region during low pH environment helps with deployment of the augmentation gel because the gel has to be delivered through a small diameter tubing. Polymers that are pH sensitive are also called polyelectrolytes. The swelling properties of polyelectrolyte networks, which can be described in terms of the swelling rate and maximum solution uptake at equilibrium, depend on the physicochemical properties of the polymers and on the composition of the surrounding medium. Polyelectrolyte gels change their conformation with the degree of dissociation which is the function of quantities such as pH value, polarity of the solvent, ionic strength and temperature of the external environment solution.

### Example C

Synthesis of biocompatible and biodegradable polyelectrolyte hydrogels based on polyvinyl pyrrolidone (PVP), gelatin and hyaluronic acid (HA) using gamma irradiation polymerization technique. The example polymers of C1 and C2 at pH 5 exponentially increased their water absorption properties. The addition of PVP and gelatin were for in-vitro handling and processing ease.

C1.

| | |
|---|---|
| PVP | 5 g |
| Hyaluronic acid | 1 g |

Mix well and expose the mixture to 30 kGy radiation

C2.

| | |
|---|---|
| Gelatin | 10 g |
| Hyaluronic acid | 1 g |

Mix well and expose the mixture to 30 kGy radiation

### Example D

Synthesis of hyaluronic acid and polyvinyl alcohol at various respective ratios in an interpenetrating networks. The polyvinyl alcohol included in the polymer system was for ease of in-vitro handling and processing. Glutaraldehyde and hydrochloric acid were catalysts for the PVA reaction. The 1-ethyl-(3-3-dimethylaminopropyl) carbodiimide hydrochloride was the catalyst for the hyaluronic acid reaction. The two materials independently crosslinked at their primary structure levels while their secondary structures intertwined to create interpenetrating polymer networks. Examples D1, D2 and D3, at pH 4 exponential changed their water absorption properties.

D1.

| | |
|---|---|
| Hyaluronic acid | 3 g |
| Polyvinyl alcohol | 1 g |

D2.

| | |
|---|---|
| Hyaluronic acid | 1 g |
| Polyvinyl alcohol | 1 g |

D3.

| | |
|---|---|
| Hyaluronic acid | 1 g |
| Polyvinyl alcohol | 3 g |

Another preferred embodiment is filling a silicone shell with cross-linked hyaluronic acid material. This method required a high sheer mixer. The HA is cross linked using available cross-linkers such as divinylsulfone, 1,4-butane dioldiglycidyl ether in the presence of 0.1M sodium hydroxide. When the crosslinking reaction has completed, the HA gel is washed repeatedly until the residual cross-linker was no longer detectable in the HA gel, At this point, the cross-linked gel is blended with 10% water in shear mode to create uniform and small particles. The blended cross-linked material reformulated with un-cross-linked materials HA for injectability and longevity.

The implants of the present invention further can be instilled, before or after implantation, with indicated medicines and other chemical or diagnostic agents. Examples of such agents include, but are not limited to, antibiotics, chemotherapies, other cancer therapies, brachytherapeutic material for local radiation effect, x-ray opaque or metallic material for identification of the area, hemostatic material for control of bleeding, growth factor hormones, immune system factors, gene therapies, biochemical indicators or vectors, and other types of therapeutic or diagnostic materials which may enhance the treatment of the patient.

The present invention has been described particularly in connection with a breast, butt, or body implant, but it will be obvious to those of skill in the art that the invention can have application to other parts of the body, such as the face, and generally to other soft tissue or bone. Accordingly, the invention is applicable to replacing missing or damaged soft tissue, structural tissue or bone, or for cosmetic tissue or bone replacement.

Although the present invention has been described in relation to particular embodiments thereof, many other variations and modifications and other uses will become apparent to those skilled in the art. It is preferred, therefore, that the present invention be limited not by the specific disclosure herein, but only by the appended claims. The other methods, used for characterization of the products according to one embodiment are described in the following examples which illustrate preferred embodiments of one embodiment without, however, being a limitation thereof. Variations and modifications can, of course, be made without departing from the spirit and scope of the invention.

What is claimed is:

1. A method for cosmetic augmentation, comprising:
storing a non-toxic biocompatible cross-linker;
storing a biocompatible polymer having a multi-phase mixture with a predetermined controlled release of selected pharmaceutical substance to modulate soft tissue response to the polymer, wherein the polymer reacts in-situ to alter physical properties of the polymer from a deformable state to a non-deformable state;
mixing the biocompatible cross-linker and polymer into a mixture;
injecting the mixture into a patient and during or after injection, cross-linking the polymer in the patient; and
augmenting soft tissue with the biocompatible cross-linked polymer.

2. The method of claim 1, comprising introducing the polymer into the shell of a soft tissue human implant prior to or during implantation of the shell with a lumen in a human body.

3. The method of claim 1, comprising cross-linking the polymer, wherein a cross linking reaction occurs outside the shell or in-situ inside the shell.

4. The method of claim 1, wherein the polymer comprises one of collagens, PEG, hyaluronic acids, celluloses, proteins, saccharides, biodegradable and bioresorbable biocompatible materials.

5. The method of claim 1, wherein the polymer comprises an extracellular matrix of a biological system.

6. The method of claim 1, comprising using cross linkers and forming homo-polymers or to form copolymers by crosslinking with other polymer species.

7. The method of claim 1, comprising adding a substance to the composition for biocompatibility.

8. The method of claim 1, comprising controlling drug releases at predetermined timing in anticipation of an onset of a negative physiological event in response to an invading foreign bodies.

9. The method of claim 1, comprising fast releasing, medium or slow releasing the composition.

10. The method of claim 1, comprising adding anesthetics, lidocaine or compound to reduce or eliminate acute inflammatory reactions to the pharmaceutical substance.

11. The method of claim 1, comprising adding one or more compositions selected from the group consisting of steroids, corticosteroids, dexamethasone, triamcinolone.

12. The method of claim 1, comprising providing an antiproliferative compound.

13. The method of claim 1, wherein the substance comprises paclitaxel, serolimas.

14. The method of claim 1, comprising controlling the scar formation process around a foreign body including capsular formation.

15. The method of claim 1, comprising optimizing degradation profile of the composition.

16. The method of claim 1, comprising minimizing migration of the composition.

17. The method of claim 1, comprising controlling the number average molecular weight (Mn) and the polydispersity index. The method of claim 1, comprising characterizing a target tissue, and maintaining a consistency of the composition in particle size and population densities.

18. The method of claim 1, comprising co-cross-linking glycosaminoglycan chemically with at least one other polymer including hyaluronan or hylan.

19. The method of claim 1, wherein the chemically cross-linked glycosaminoglycan is hyaluronan or hylan.

20. The method of claim 1, comprising in-situ non-covalent bonding including one of: hydrogen associatation, charge or ionic interactions, pH, osmolality.

21. The method of claim 1, comprising modeling a 3D model of a human body and continuously updating a current shape of breast or butt from the 3D model to fit to a desired shape.

22. The method of claim 1, comprising injecting with a mechanical pump the biocompatible crosslinked polymer under soft tissue in a minimally invasive manner.

23. A method for cosmetic augmentation, comprising:
storing a non-toxic biocompatible cross-linker;
storing a biocompatible polymer having a multi-phase mixture with a predetermined controlled release of selected pharmaceutical substance to modulate soft tissue response to the polymer;
mixing the biocompatible cross-linker and polymer into a mixture;
injecting the mixture into a patient and during or after injection, cross-linking the polymer in the patient; and
augmenting soft tissue with the biocompatible cross-linked polymer.
